(19) United States
(12) Patent Application Publication
(10) Pub. No.: US 2015/0141793 A1
(43) Pub. Date: May 21, 2015

(54) METHOD OF TRACKING AN AFFECTED AREA AND A SURGICAL EQUIPMENT

(71) Applicants: KYUNGPOOK NATIONAL UNIVERSITY INDUSITRY-ACADEMIC COOPERATION FOUNDATION, Daegu (KR); KOH YOUNG TECHNOLOGY INC., Seoul (KR)

(72) Inventors: Jong-Kyu Hong, Gwangju-si (KR); Hyun-Ki Lee, Daegu (KR); Min-Young Kim, Daegu (KR); Jae-Heon Chung, Gwangmyeong-si (KR)

(73) Assignee: KYUNGPOOK NATIONAL UNIVERSITY INDUSTRY-ACADEMIC COOPERATION FOUNDATION, Daegu (KR)

(21) Appl. No.: 14/241,959

(22) PCT Filed: Apr. 19, 2013
(86) PCT No.: PCT/KR2013/003355

§ 371 Date: Feb. 28, 2014

(30) Foreign Application Priority Data
Apr. 27, 2012 (KR) 10-2012-0044787

(51) Int. Cl. A61B 19/00

(52) U.S. Cl. CPC A61B 19/54; A61B 19/5223; A61B 19/5225; A61B 2019/5445; A61B 2019/5458
USPC 600/407

(57) ABSTRACT

A method of tracking an affected area and a surgical equipment, which is capable of tracing positions of the affected area and the surgical equipment more precisely through a stereo scope by using images of the affected area and the surgical equipment traced in macro scale after tracing the affected area and the surgical equipment in macro scale. The method of tracking an affected area and a surgical equipment, includes a step of macro tracking in which energy emitted from a plurality of markers attached to the affected area and the surgical equipment is sensed to trace positions of the affected are and the surgical equipment; a step of image input in which images of the affected area and the surgical equipment that are traced in the step of macro tracking step are captured by the tracking sensor and the images of the affected area and the surgical equipment , which are captured by the tracking sensor, are inputted to a stereo display part of a microscope; and a step of micro tracking in which the positions of the affected area and the surgical equipment are traced based on a coordinate of the microscope by using macro images of the stereo display part of the microscope.

### TECHNICAL FIELD

The present invention relates to a method of tracking an affected area and a surgical equipment, and more particularly to a method of tracking an affected area and a surgical equipment by using a tracking sensor, marker and a stereo microscope.

### BACKGROUND ART

In general, in order to detect a penetrating device such as a catheter and a surgical equipment and an affected area of a body in surgical operation, a tracking device is used.

The tracking device includes a plurality of markers attached to a surgical equipment and an affected area, a tracking sensor sensing the markers, and a processor connected to the tracking sensor in order to determine the position of the markers.

According to a conventional tracking method using the tracking device, the tracking sensor senses energy emitted by the plurality of markers, and the processor determines the position of energy emitted by the markers and sensed by the tracking sensors, and matches positions of the energy of the sensed markers with previously set markers corresponding to the markers to trace the markers so that the position of the surgical equipment and the affected area.

However, according to the conventional tracking method tracing a surgical equipment and an affected area, the energy emitted by the markers is sensed to trace the position of the surgical equipment and the affected area so that the position is roughly detected. Therefore, more precise method of tracking a surgical equipment and an affected area is required.

### DETAILED DESCRIPTION OF THE INVENTION

### Objects of the Invention

Therefore, the object of the present invention is to provide a method of tracking an affected area and a surgical equipment, which is capable of precisely detecting the position of a surgical equipment and an affected area.

### Technical Solution

A method of tracking an affected area and a surgical equipment, includes a step of macro tracking in which energy emitted from a plurality of markers attached to the affected area and the surgical equipment is sensed to trace positions of the affected are and the surgical equipment; a step of image input in which images of the affected area and the surgical equipment that are traced in the step of macro tracking step are captured by the tracking sensor and the images of the affected area and the surgical equipment , which are captured by the tracking sensor, are inputted to a stereo display part of a microscope; and a step of micro tracking in which the positions of the affected area and the surgical equipment are traced based on a coordinate of the microscope by using macro images of the stereo display part of the microscope.

### Advantageous Effects

According to the method of tracking an affected area and a surgical equipment, energy emitted from a plurality of markers and attached to the affected area and the surgical equipment is sensed through a tracking sensor to trace in macro scale, images of the affected area and the surgical equipment of which positions are traced in macro scale, are captured by the tracking sensor to input to the stereo display part of a microscope, and the positions of the affected area and the surgical equipment are more precisely traced based on the coordinate of the stereo microscope through the stereo microscope by using macro image of the affected area and the surgical equipment of which positions are traced in macro scale so that more safe and precise operation may be performed.

### BRIEF DESCRIPTION OF THE DRAWINGS

FIG. 1 is a view for explaining a tracking method according to an exemplary embodiment of the present invention.

FIG. 2 is a block diagram for explaining a tracking method according to an exemplary embodiment of the present invention.

FIG. 3 is a block diagram for explaining a step of macro tracking.

FIG. 4 is a block diagram for explaining a step of image input.

### EMBODIMENTS OF THE INVENTION

This invention may be embodied in many different forms, and will be described with reference to the accompanying drawings. But this invention should not be construed as limited to the embodiments set forth herein, but should be understood to include every modifications, equivalents and substitutes

The terms such as ‘first’, ‘second’, etc. may be used for various elements but the elements should not limited by the terms. The terms may be used only for discriminating one element from others. For example, a first element may be named as a second element, and the second element may be named as the first element within the present invention.

The terms used in the present application are only to explain the specific embodiment and is not intended to limit the present invention. The terms “a”, “an” and “the” mean “one or more” unless expressly specified otherwise. The terms “including”, “comprising”, etc., are to designate features, numbers, processes, structural elements, parts, and combined component of the application, and should be understood that it does not exclude one or more different features, numbers, processes, structural elements, parts, combined component.

The technical term or the scientific term that will be used in the specification has to the same meaning as a person skilled in the art commonly understood unless defined differently.

The terms defined in a commonly used dictionary should be understood as the context, and should not be understood ideally or excessively unless defined differently.

Hereinafter, preferred embodiments of the present invention will be explained referring to figures.

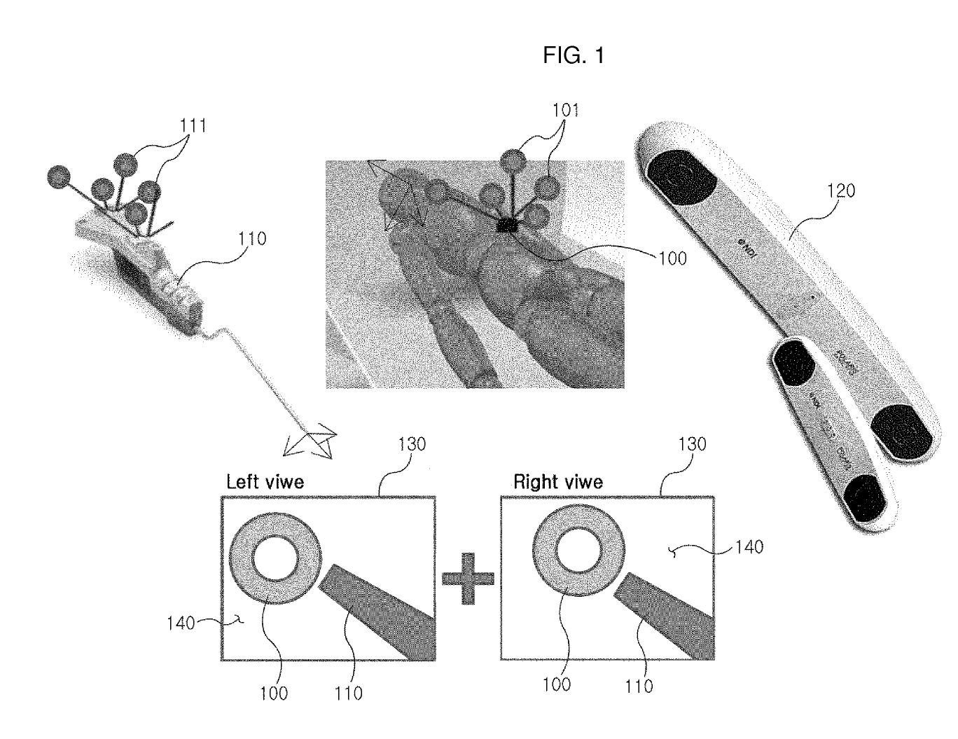

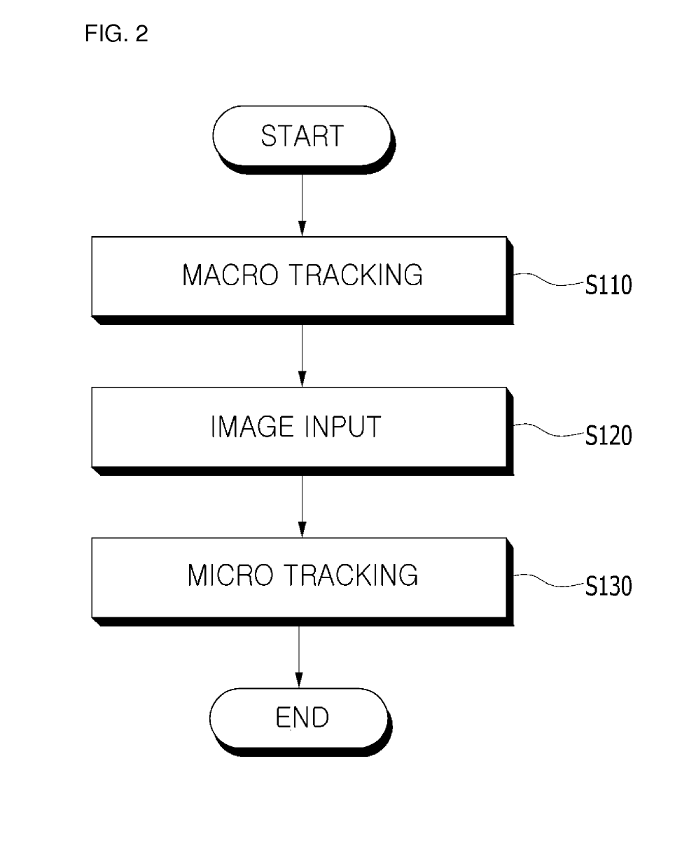

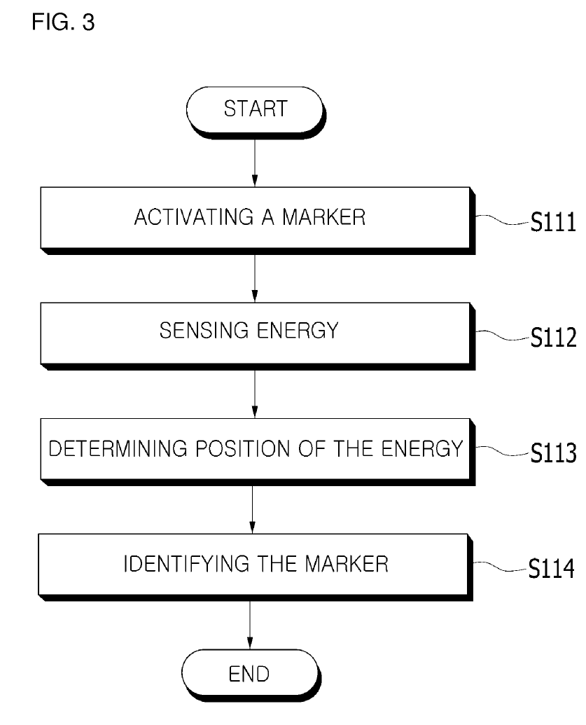

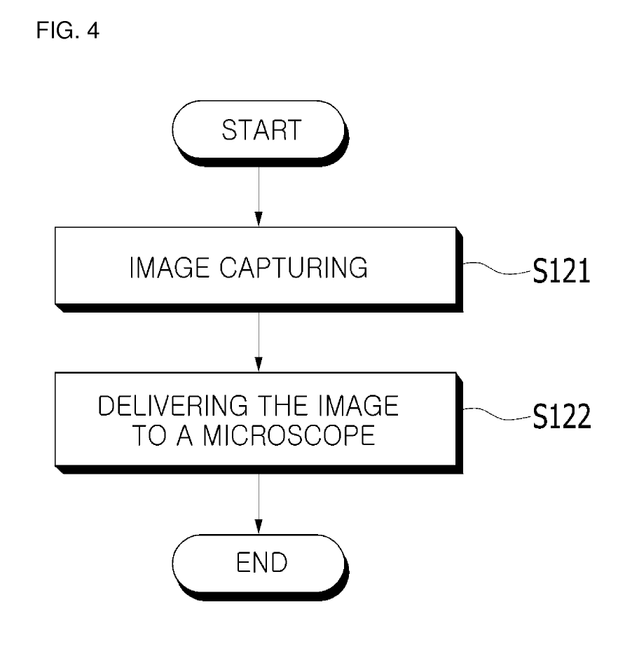

FIG. 1 is a view for explaining a tracking method according to an exemplary embodiment of the present invention, FIG. 2 is a block diagram for explaining a tracking method according to an exemplary embodiment of the present invention, FIG. 3 is a block diagram for explaining a step of macro tracking, and FIG. 4 is a block diagram for explaining a step of image input.

Referring to FIG. 1 and FIG. 2, a tracking method according to an exemplary embodiment of the present invention includes a step of macro tracking (S110), a step of image input (S120), and a step of micro tracking (S130).

In the step of macro tracking (S110), a tracking sensor (120) senses energy emitted from a plurality of markers 111 and 101 attached to an affected area 100 and a surgical equipment 110, and a processor (not shown) determines the position of the affected area (100) and the surgical equipment (110).

In detail, the step of macro tracking (S110) will be explained referring to FIG. 3.

Referring to FIG. 3, the step of macro tracking (S110) includes a step of activating a marker (S111), a step of sensing energy (S112), a step of determining position of the energy (S113), and a step of identifying the marker (S114).

In the step of activating a marker (S111), the plurality of markers 111 and 101 attached to the affected area 100 and the surgical equipment 110 are activated by the processor. In this case, each of the markers 111 and 101 attached to the affected area 100 and the surgical equipment 110 may emit light by itself or reflect external light. Alternatively, each of the markers 111 and 101 may generate magnetic field.

In the step of sensing energy (S112), when the markers 111 and 101 are activated, a tracking sensor 120 senses the energy emitted by the activated markers 111 and 101.

In the step of determining position of the energy (S113), when the energy is sensed by the tracking sensor 120, the processor determines the position of the energy emitted from the markers 111 and 101 of which energy is sensed by the tracking sensor 120.

In step of identifying the marker (S114), the processor matches the markers 111 and 101 of which energy is sensed with previously set markers that are previously set in the processor and correspond to the marker to trace the sensed markers 111 and 101 so that the positions of the surgical equipment 110 and the affected area 100 are traced in macro scale.

Referring again to FIG. 1 and FIG. 2, in the step of image input (S120), images of the affected area 100 and the surgical equipment 110 traced by the tracking sensor 120 in the step of macro tracking (S110), are captured, and the captured images are inputted to the stereo display part 130 of a microscope by the processor.

In detail, the step of image input (S120) will be explained referring to FIG. 4.

Referring to FIG. 4, the step of image input (S120) includes a step of image capturing (S121) and a step of delivering the image to a microscope (S122).

In the step of image capturing (S121), the images of the affected area 100 and the surgical equipment 110 that are traced in the step of macro tracking (S110), are captured by the tracking sensor 120 activated by the processor, and the captured images of the affected area 100 and the surgical equipment 110 are inputted to the processor.

In the step of delivering the image to a microscope (S122), the images of the affected area 100 and the surgical equipment 110, which are captured by the tracking sensor 120 is image processed by the processor, and the processor delivers the processed image to a stereo display part 130 of a stereo microscope.

Referring again to FIG. 1 and FIG. 2, in the step of micro tracking (S130), the positions of the affected area 100 and the surgical equipment 110 are more precisely traced based on a microscope coordinate through a macro image 140 of the affected area 100 and the surgical equipment 110, which is inputted into the stereo display part 130 of the microscope in a macro scale. That is, when the image of the affected area 100 and the surgical equipment 110 is inputted to the stereo display part 130 of the microscope, which is captured by the tracking sensor 120, the image of the affected area 100 and the surgical equipment 110 may be observed through ocular lenses for both eyes as shown in FIG. 1, so that the positions of the affected area 100 and the surgical equipment 110 may be more exactly and precisely traced based on the microscope coordinate by using the stereo microscope.

As described above, according to the method of tracking an affected area 100 and a surgical equipment 110, energy emitted from a plurality of markers 111 and 101 attached to the affected area 100 and the surgical equipment 110 is sensed through a tracking sensor 120 to trace in macro scale, images of the affected area 100 and the surgical equipment 110 of which positions are traced in macro scale, are captured by the tracking sensor 120 to input to the stereo display part 130 of a microscope, and the positions of the affected area 100 and the surgical equipment 110 are more precisely traced based on the coordinate of the stereo microscope to through the stereo microscope by using macro image 140 of the affected area 100 and the surgical equipment 110 of which positions are traced in macro scale.

The detailed description of the present invention is described with regard to the preferable embodiment of the present invention, however, a person skilled in the art may amend or modify the present invention within the spirit or scope in the following claim of the present invention.

What is claimed is:

1. A method of tracking an affected area and a surgical equipment, the method comprising:
a step of macro tracking in which energy emitted from a plurality of markers attached to the affected area and the surgical equipment is sensed to trace positions of the affected are and the surgical equipment;
a step of image input in which images of the affected area and the surgical equipment that are traced in the step of macro tracking step are captured by the tracking sensor and the images of the affected area and the surgical equipment, which are captured by the tracking sensor, are inputted to a stereo display part of a microscope; and
a step of micro tracking in which the positions of the affected area and the surgical equipment are traced based on a coordinate of the microscope by using macro images of the stereo display part of the microscope.
